(19) United States
(12) Patent Application Publication
(10) Pub. No.: US 2019/0175671 A1
(43) Pub. Date: Jun. 13, 2019

(54) MICRORNA 22 REGULATORS COMPOSITIONS AND METHODS AND USES THEREOF

(71) Applicant: Phaethon Psychis, Athens (GR)

(72) Inventor: Phaethon Psychis, Athens (GR)

(21) Appl. No.: 16/213,267

(22) Filed: Dec. 7, 2018

Related U.S. Application Data
(60) Provisional application No. 62/596,752, filed on Dec. 8, 2017.

(51) Int. Cl. A61K 35/76; A61K 31/713

(52) U.S. Cl. CPC A61K 35/76; A61K 31/713

(57) ABSTRACT

This disclosure generally relates to microRNA 22 regulators compositions and methods and uses thereof for treatment of diseases related to the function and/or expression of microRNA 22. Such diseases include cancer, neurodegenerative diseases, inflammatory diseases, and viral infection.

This application claims the benefit of U.S. provisional application 62/596,752 filed Dec. 8, 2017.

### TECHNICAL FIELD

This disclosure generally relates to microRNA 22 regulators compositions and methods and uses thereof for treatment of diseases related to the function and/or expression of microRNA 22.

### BACKGROUND

MicroRNAs (abbreviated miRNA) are small non-coding RNA molecules (containing about 22 nucleotides) found in plants, animals and some viruses, that function in RNA silencing and post-transcriptional regulation of gene expression. In particular, microRNA 22 has been connected to many physiological functions, normal and disease-related, including tumorigenesis, epigenetic modification, embryonic development, cardiac disorders, skeletal metabolism, and various psychological disorders. Of special interest is microRNA 22 involvement in inflammatory processes and diseases. Inflammation is the response of body tissues to harmful stimuli, such as pathogens, damaged cells, or irritants. Inflammatory diseases include cancer, atherosclerosis, and ischemic heart disease. Regulation of microRNA 22 expression and/or function present a new and effective approach for treatment of various diseases.

### SUMMARY

In some embodiments, the present disclosure provides a method for altering the Interferon signaling pathway in a cell comprising contacting the cell with a compound or a molecule that alters the of one or more of Interferon Regulatory Factors IRF1. IRF2, IRF3, IRF4, IRF5, IRF6, IRF7, IRF8, IRF9 or homologues thereof, or the activity, of an encoded protein, in the cell. In some embodiments, the molecule is an enhancer of the Interferon signaling pathway in a cell and enhances the expression of Interferon Regulatory Factor 9 (IRF9) or homologues thereof, or the activity of an encoded protein, in the cell.

In some embodiments, the said compound or a molecule is inhibiting the interferon signaling and is selected from one of the groups, lists, or descriptions of the present disclosure. In some embodiments, the said inhibitory molecule or compound is: a modified vaccinia virus synthetically modified by the presence, in a non-essential region of the vaccinia genome, of DNA not naturally occurring in vaccinia virus, wherein said DNA is expressed in a host by the production of a protein, and wherein said protein is an antigen.

In some embodiments, the present disclosure provides a method for increasing survivability of a cell comprising administering to the cell an amount of a compound or molecule that inhibits the expression of Hn-I 4, lin-28, lin-42, egl-35 or homologues thereof, or the activity of an encoded protein, in the cell. In some embodiments, the compound or molecule is selected from one of the groups lists, or descriptions of the present disclosure.

In some embodiments, the compound or molecule is administered to, or expression is increased in, the cells of, an individual for treatment or prevention of an age-related disorder or premature aging, and wherein the compound or molecule is selected from one of the groups lists, or descriptions of the present disclosure. In some embodiments, the age-related disorder is selected from the group consisting of Alzheimer's, Parkinson's, diabetes, dementia, atherosclerosis, arthritis, stroke, high blood pressure, and heart disease. In some embodiments, the individual is a human.

In some embodiments, the present disclosure provides a method for altering the Interferon signaling pathway in a cell comprising contacting the cell with a compound or a molecule that alters expression of one or more of Interferon Regulatory Factors IRF1. IRF2, IRF3, IRF4, IRF5, IRF6, IRF7, IRF8, IRF9 or homologues thereof, or the activity, of an encoded protein, in the cell. In some embodiment, the said compound or molecule is an enhancer of the Interferon signaling pathway in a cell and enhances the expression of Interferon Regulatory Factor 9 (IRF9) or homologues thereof, or the activity of an encoded protein, in the cell, wherein the compound or molecule is selected from one of the groups lists, or descriptions of the present disclosure. In some embodiments, the inhibitory molecule is administered to, or expression is increased in, the cells of, an individual for treatment or prevention of an inflammatory disorder. In some embodiments, the inflammatory disorder is selected from the group consisting of allergic reactions, myopathies, abnormal inflammation, atherosclerosis, ischemic heart disease, Rheumatic fever, and Rheumatoid arthritis. In some embodiments, the individual is a human.

In some embodiments, the present disclosure provides a method for altering the Interferon signaling pathway in a cell comprising contacting the cell with a compound or molecule or a molecule that alters expression of one or more of Interferon Regulatory Factors IRF1. IRF2, IRF3, IRF4, IRF5, IRF6, IRF7, IRF8, IRF9 or homologues thereof, or the activity, of an encoded protein, in the cell, for increasing survivability of a cell. In some embodiments, the said method comprises administering to the cell an amount of a compound or molecule that inhibits the expression of Interferon Regulatory Factors or homologues thereof, or the activity of an encoded molecule, in the cell, wherein the said compound or molecule is selected from one of the groups lists, or descriptions of the present disclosure. In some embodiments, the inhibitory molecule is administered to, or expression is increased in, the cells of, an individual for treatment or prevention of cancer. In some embodiments, the individual is a human.

In any of the methods of the disclosure, the composition may be administered to the subject by any method known in the art (e.g., those described herein). In certain embodiments, the composition is administered by a route selected from the group consisting of oral, rectal, parenteral (e.g., intravenously or intramuscularly), cutaneous, topical, nasal, vaginal, inhalant, skin (patch), and ocular.

By “treating” is meant ameliorating at least one symptom of a condition or disease in a subject having the condition or disease (e.g., a subject diagnosed with a neurological disorder), as compared with an equivalent untreated control. Such reduction in the symptom (e.g., a reduction in blood glucose levels) is at least 5%, 10%, 20%, 40%, 50%, 60%, 80%, 90%, 95%, or 100%, as measured by any standard technique.

By “treating prophylactically” is meant to reduce the frequency of disease occurrence or severity of disease upon its onset by administering to the subject a therapeutic prior to onset of the disease. Prophylactic treatment can include disease prevention. Subjects at higher risk of developing metabolic disorders or IBD (e.g., risk factors described herein) may be treated prophylactically in the methods of the disclosure.

By “neurological disorder” is meant any disease, condition and disorder of the central nervous system. Neurological disorders according to the present disclosure are, but not limited to, Alzheimer's disease, Parkinson's Disease, amyotrophic lateral sclerosis, epilepsy, autism, rett syndrome, Huntington's Disease, stroke, spinal cord injury, traumatic brain injury, Lewy body dementia, multiple sclerosis, Pick's disease, Niewmann-Pick disease, Creutzfeld-Jakob disease, Guillain-Barre syndrome, Bell's palsy, diabetic neuropathy, amyotrophic lateral sclerosis, amyloid angiopathy, cerebral amyloid angiopathy, systemic amyloidosis, hereditary cerebral hemorrhage with amyloidosis of the Dutch type, inclusion body myositis, mild cognitive impairment, complications due to stroke, head trauma, or spinal injury, or other injuries to the brain, peripheral nervous, central nervous, or neuromuscular system, acute spinal cord and brain injury, and demyelinating diseases, such as multiple sclerosis.

By “effective amount” is meant an amount of a compound or molecule required to treat, treat prophylactically, or reduce disease or disorder in a clinically relevant manner. For example, a effective amount of an active compound or molecule used to according to the present disclosure varies depending upon the manner of administration, the age, body weight, and general health of the subject. Ultimately, the prescribers will decide the appropriate amount and dosage regimen.

By “subject” is meant a human or non-human animal (e.g., a mammal).

By “increase” is meant an amount greater by at least 5%, 10%, 25%, 50%, 100%, 150%, 200%, 500%, or 1000%.

### DETAILED DESCRIPTION

Chronic inflammation is a hallmark of more than 80 recognized autoimmune disorders, and the site of the symptoms (joints, skin, organs) determines the specific disorder. Chronic inflammation is not a specific disease but a mechanistic process. The diseases associated with chronic inflammation are multiple and include CVD, diabetes, malignancy, auto-immune disease, chronic hepatic, renal disease, Chronic kidney disease (CKD), Allergic asthma, Chronic obstructive pulmonary disease (COPD), certain CNS neurodegenerative diseases such as Dementia and cognitive decline and also acute inflammation. It appears that Alzheimer's disease may be another example of an autoimmune disease in which brain inflammation plays a causative role.

Chronic inflammatory diseases are the most significant cause of death in the world. The World Health Organization (WHO) ranks chronic diseases as the greatest threat to human health. The prevalence of diseases associated with chronic inflammation is anticipated to increase persistently for the next 30 years in the United States. In 2000, nearly 125 million Americans were living with chronic conditions and 61 million (21%) had more than one. In 2014 nearly 60% of Americans had at least one chronic condition, 42% had more than one and 12% of adults had 5 or more chronic conditions. Worldwide, 3 of 5 people die due to chronic inflammatory diseases like stroke, chronic respiratory diseases, heart disorders, cancer, obesity, and diabetes.

Chronic inflammation of the body's peripheral systems may also lead to brain inflammation and Alzheimer's disease. Long term gum disease, for instance, has been linked to increased risk of developing Alzheimer's disease.

Development of Inflammation of the brain is similar to the inflammatory processes that take place in the rest of the body. Microglia in the dedicated immune system of the central nervous system are activated when they perceive a threat, and are dispatched in response to promote healing. However, when microglia are constantly responding to perceived threats, the immune system is overactivated and this can lead to damage of healthy brain cells and acceleration of the Alzheimer's disease process. Microglia activation has a negative effect on normal cognitive function.

Ribonucleic acid is a polymeric molecule essential in various biological roles in coding, decoding, regulation and expression of genes. RNA and DNA are nucleic acids, and, along with compounds, proteins and carbohydrates, constitute the four major macromolecules essential for all known forms of life.

MicroRNAs generally function in RNA silencing and post-transcriptional regulation of gene expression. MicroRNAs function via base-pairing with complementary sequences within mRNA molecules. As a result, these mRNA molecules are silenced, by one or more of the following processes: (1) Cleavage of the mRNA strand into two pieces, (2) Destabilization of the mRNA through shortening of its poly(A) tail, and (3) Less efficient translation of the mRNA into proteins by ribosomes.

MIR22HG is a long non-coding (lnc) RNA highly expressed in human cells in some diseases or stress conditions. Interestingly, lncRNA MIR22HG hosts, in the second exon, the microRNA 22 (also known as, and referred to herein, as MIR22 or miR-22). MicroRNAs (miRNAs), such as MIR22, are noncoding RNAs of 18 to 24 nucleotides that regulate the stability and translational efficiency of complementary target mRNAs. Chang et al., (2008) mapped the MIR22 gene to an exon of the MIR22HG (C17ORF91) gene. MicroRNA 22 is a circadian rhythm oscillating MicroRNA.

MicroRNA 22 has been connected to many physiological functions, normal and disease-related, including tumorigenesis, epigenetic modification, embryonic development, cardiac disorders, skeletal metabolism, and various psychological disorders.

### MicroRNA 22 Mode of Action

Recently, extensive evidence has demonstrated that microRNA 22 could repress tumor malignant process by inhibition of the cell cycle. For example, microRNA 22 may post-transcriptionally target cyclin A2 and CDKN1A (cyclin-dependent kinase inhibitor 1A) to arrest the cell cycle in G0/G1 phage in CRC and liver cancer, respectively. Besides, augmenting expression of microRNA 22 in ER (estrogen receptor) α-positive endometrioid adenocarcinoma where microRNA 22 expression is usually low could downregulate ERα expression to further decrease the expression of cyclin D1 and member matrix metalloproteinase 2/9. Furthermore, carcinogen TPA (12-O-tetradecanoylphorbol-13-acetate)-induced microRNA 22 may inversely regulate PKC/ERK pathway via dramatically downregulating Max (a transcription factor binding to and activate c-Myc) expression, thus resulting in G0/G1 arrest in lung, breast and prostate cancer cells. These findings confirmed that the ultimate effects of microRNA 22 by cell cycle arrest in different ways may lead to the attenuation of cancer growth and invasion and the disruption of tumor malignant progression. Specifically, through targeting various downstream related molecules, such as HIF-1α, GLUT1, ACLY, SIRT1, CDK6, Sp1, CD151, MTDH, Galectin-9, NET1, PAPST1, ESR1, TIAM1, Max, Cyclin A2/CDKN1A, Erbb3, EVI-1, PTEN and ERα, microRNA 22 is capable of directly or indirectly abrogate the process of tumor malignancy, including acceleration of senescence and the abruption of angiogenesis, energy metabolism, cell cycle, proliferation, migration, invasion and metastasis (Wang et al., 2017).

Other results also suggest that microRNA 22-mediated regulation plays an important role in the mechanisms of action of IFN-beta, including in the treatment of multiple sclerosis and normal immune responses (Hecker et al., 2013). Further, it was found that microRNA 22 is induced in glial cells upon stimulation with poly(I:C). Overexpression of microRNA 22 in the cultured cells resulted in decreased activity of interferon regulatory factor-3 and nuclear factor-kappa B, which in turn led to reduced expression of interferon-β and inflammatory cytokines, including tumor necrosis factor-α, interleukin-1β, interleukin-6, and chemokine (C-C motif) ligand 5, whereas knockdown of microRNA 22 had the opposite effect. It was demonstrated that mitochondrial antiviral signaling protein (MAVS), which positively regulates type I interferon production, is a novel target of microRNA 22. Thus, microRNA 22 negatively regulates poly(I:C)-induced production of type I interferon and inflammatory cytokines via targeting MAVS (Wan et al., 2016).

In addition, it was found that microRNA 22 suppresses interferon gene expression by directly targeting high mobility group box-1 and interferon regulatory factor (IRF)-5, preventing activation of IRF3 and NF-κB, which are activators of interferon genes. The expression of interferon genes is elevated in quiescent cells and their expression is inhibitory for cell proliferation. Further, microRNA 22 was found to be activated by the transcription factor Myc when quiescent cells enter proliferation, and it was found that microRNA 22 inhibits the Myc transcriptional repressor MXD4, mediating a feed-forward loop to elevate Myc expression levels. This implicates microRNA 22 in downregulating the anti-proliferative p53 and interferon pathways and reveal a new transcription factor—miRNA network that regulates the transition of primary human cells from quiescence to proliferation. Both IRF5 and HMGB1 (high mobility group box-1) mediate interferon signaling pathways. IRF5 is involved in the induction of proinflammatory cytokines in response to several different Toll-like receptor (TLR) ligands and mediates signaling downstream of the TLR7 and TLR8 pathways to activate the interferon-beta promoter. HMGB proteins are highly represented in the nucleus and are responsible for regulating transcription and chromatin structure. However, HMGB1 was recently shown to act as a universal sensor for double-stranded RNA as well as DNA and stimulate the interferon pathway by activating the transcription factors IRF3 and NF-κB, which are essential components of the interferon-beta promoter ‘enhanceosome’ (Polioudakis et al., 2013). Human interferon-beta, a crucial component of innate antiviral immunity, is upregulated in cells that are infected by viruses, thus modulating the expression of microRNA 22 may affect the human body response to infection by viruses. On the other hand, chronic over-expression of interferon-beta may result in chronic inflammation and associated diseases.

### The Transcriptional Switch

The IFNβ gene promoter contains binding sites for IRF3, NF-κB, and AP-1 that form the enhanceosome enabling efficient transcription of the IFNβ gene (Maniatis et al., 1998; Thanos and Maniatis, 1995).

Activation of interferon-beta transcription is a highly ordered process beginning with the delivery of NF-kappaB to the IFN-beta enhancer through a process involving stochastic interchromosomal interactions between the IFN-beta enhancer and specialized Alu elements. NF-kappaB delivery is followed by the binding of ATF-2/c-Jun and IRF proteins in a highly cooperative fashion. The assembled “enhanceosome” then recruits PCAF/GCN5 which acetylates the histone tails of the adjacent nucleosomes. The transcriptional coactivator CBP, which binds in a complex with the RNA polymerase II holoenzyme is recruited by the enhanceosome replacing PCAF/GCN5. Next, SWI/SNF, which is part of the holoenzyme complex, induces a conformational change in a nucleosome positioned over the transcriptional start site allowing TFIID to bind, which promotes the sliding of this nucleosome to a new downstream position. At this point the full pre-initiation complex is assembled and transcription commences. This detailed picture of the IFN-beta transcription program gathered through years of rigorous studies, now serves as a paradigm for understanding complex transcriptional switches in eukaryotic systems (Ford and Thanos, 2010).

### The IRF9-STAT2/ISGF3 Switch

Interferon regulatory factor 9 (IRF9) is an integral transcription factor in mediating the type I interferon antiviral response, as part of the interferon-stimulated gene factor 3 (ISGF3). However, the role of IRF9 in many important non-communicable diseases has just begun to emerge. The duality of IRF9's role in conferring protection but at the same time exacerbates diseases is certainly puzzling. The regulation of IRF9 during these conditions is not well understood. The high homology of IRF9 DNA-binding domain to other IRFs, as well as the recently resolved IRF9 IRF-associated domain structure can provide the necessary insights for progressive inroads on understanding the regulatory mechanism of IRF9. In the canonical JAK-STAT pathway, binding of type I IFNs to its receptors (IFNAR1 and IFNAR2) leads to the dimerization of both IFNARs. This in turn phosphorylates IFNAR1-bound tyrosine kinase 2 (TYK2) which then phosphorylates IFNAR2-bound Janus kinase 1 (JAK1). Then, the receptor-bound kinases phosphorylate STAT1 and STAT2 at amino acid position 701 and 690, respectively. The phosphorylated STAT1 and STAT2 subsequently dimerizes via reciprocal SH2-phosphotyrosine interactions. Phosphorylated STAT1-STAT2 heterodimer then dissociates from the receptors and recruit IRF9 to form the ISGF3 complex in cytoplasm. ISGF3 will translocate into the nucleus and binds to the promoter region of interferon-stimulated response element (ISRE) to activate the transcription of interferon stimulated genes (Paul et al., 2018).

### Chronic Pain and Inflammation

Inflammation constitutes an essential part of the innate immune response to pathogens or the release of self molecules acting as endogenous danger signals. Exposure of peripheral tissue to pathogen- or danger-associated molecular patterns (PAMPs and DAMPs, respectively) stimulates the release of proinflammatory mediators by tissue-resident cells that activate the endothelium of blood vessels and initiate a chain of events that ends with the transmigration of blood leukocytes and their penetration of the infected or otherwise irritated tissue. The strength and persistence of the proinflammatory stimulus decides whether systemic responses such as the mobilization of bone marrow leukopoiesis or the liver acute phase response ensues. The potentially harmful consequences of the inflammatory response need to be tightly controlled. Otherwise inflamed tissue may be irreversibly damaged as a consequence of lytic enzyme release or through oxidative stress. Moreover, an overshooting systemic response may cause a generalized shock syndrome. Consequently, the outcome of the innate response to infection is determined by the balance between microbicidal effects and the damage inflicted to the host organism by the inflammation-induced loss of cell, tissue or organ function. A large number of cytokines and chemokines that regulate the generation, trafficking and effector activity of leukocytes forming the inflammatory cell infiltrate control inflammation (Rauch, 2013).

In some cases, the predominant effect of these cytokines is clearly proinflammatory, as in the case of TNF, or predominantly anti-inflammatory as in the case of TGF-β or IL-10. In other cases, cytokines may act to support or suppress inflammation, depending on context. Interferons (IFN) are frequent contributors to the inflammatory cytokine stew.

Interferons (IFNs) are secreted glycoproteins that are produced by cells in response to virus infection and other stimuli and induce an antiviral state in cells bearing IFN receptors. In this way, IFNs restrict virus replication and spread before an adaptive immune response is developed. Viruses are very sensitive to the effects of IFNs and consequently have evolved many strategies to interfere with interferon. This is particularly well illustrated by poxviruses, which have large dsDNA genomes and encode hundreds of proteins. Vaccinia virus is the prototypic poxvirus and expresses many proteins that interfere with IFN and are considered in this review. These proteins act either inside or outside the cell and within the cytoplasm or nucleus. They function by restricting the production of IFN by blocking the signaling pathways leading to transcription of IFN genes, stopping IFNs binding to their receptors, blocking IFN-induced signal transduction leading to expression of interferon-stimulated genes (ISGs), or inhibiting the antiviral activity of ISG products (Geoffrey et al 2018).

### Vaccinia Virus is an Interferon Beta Inhibitor

The role of Vaccinia Virus as an Interferon beta inhibitor has been well documented in Geoffrey L. Smith, Callum Talbot-Cooper, Yongxu Lu, Chapter Fourteen: How Does Vaccinia Virus Interfere with Interferon? Editor(s): Margaret Kielian, Thomas C. Mettenleiter, Marilyn J. Roossinck, Advances in Virus Research, Academic Press, Volume 100, 2018, Pages 355-378, hereby incorporated in its entirety by reference.

The use of Vaccinia Virus as a vector for delivery of genetic material, as well as certain synthetically modified recombinant vaccinia virus, is also well documented in the U.S. Pat. No. 4,603,112 A which is hereby incorporated in its entirety by reference.

Is microRNA 22 Associated with Super-Enhancers?

The transcriptional regulation of genes determines the fate of animal cell differentiation and subsequent organ development. With the recent progress in genome-wide technologies, the genomic landscapes of enhancers have been broadly explored in mammalian genomes, which led to the discovery of novel specific subsets of enhancers, termed super-enhancers. Super-enhancers are large clusters of enhancers covering the long region of regulatory DNA and are densely occupied by transcription factors, active histone marks, and co-activators. Accumulating evidence points to the critical role that super-enhancers play in cell type-specific development and differentiation, as well as in the development of various diseases (Shin H Y, 2018). Genes that encode candidate master transcription factors and non-coding RNAs such as miRNAs are among those associated with super-enhancers and may function to control super-enhancer activity. This is discussed in detail in patent application US20160237490)

Diseases Related to Aberrant microRNA 22 Expression or Function

Recent studies show abnormal microRNA 22 expression in cancerous tumors. MicroRNA 22 was originally identified in HeLa cells (an immortal cell line derived from cervical cancer cells), and was found to be over-expressed in prostate cancer but down-regulated in breast cancer, colon cancer, cholangiocarcinoma, multiple myeloma and hepatocellular carcinoma (Zhang et al., 2010). Mir-22 expression was associated with survival in multiple breast cancer datasets (Lánczky et al., 2016). In fact, the expression of microRNA 22 is consistently downregulated in metastatic breast cancer cells. Thus, microRNA 22 may function as a tumor suppressor. The anti-tumor properties of miR-22 was first to be demonstrated in curcumin (diferuloylmethane) research. Curcumin, a nature flavinoid derived from the rhizome of Curcuma longa, is a promising chemotherapeutic drug candidate for its low toxicity and robust anti-tumor activity in preclinical trials. However, the mechanism by which curcumin inhibits tumor growth is still obscure. In a recent research it was reported that the deleterious effect of curcumin on pancreatic cancer cells can be ascribed, at least in part, to its upregulation of microRNA 22 that reduced the expression of ER alpha and Sp1 protein. MicroRNA 22 aberrant expression is also associated, among other possible conditions, with osteoarthritic, panic Disorder, cardiomyocyte hypertrophy, pulmonary arterial hypertension, muscular disorders (Xiong et al., 2012).

In addition, as microRNA 22 also functions as a regulator of human interferon-beta gene, and its expression prevents over expression of the interferon-beta protein, aberrant expression of microRNA 22 may be related to chronic inflammation diseases. Such diseases include CNS inflammation, spinal cord inflammation, cardiovascular diseases, chronic pain, Diabetes, Arthritis and Joint Diseases, Allergies and Chronic Obstructive Pulmonary Disease (COPD). MicroRNA 22 also has multipartite anti-neurodegenerative activities including the inhibition of apoptosis and the targeting of mRNAs implicated in the etiology of neurodegenerative diseases, such as Huntington's disease (Jovicic et al., 2013).

### A Need in the Field

With cancers, neurodegenerative diseases, and chronic inflammation disorders becoming an unbearable global health and financial burdens, there is a need for a new medical approach. The involvement of MicroRNA 22 in at least the above-mentioned conditions makes it a desirable drug candidate.

An example of inadequate existing medication is the use of Recombinant Interferon treatment. Recombinant interferon treatment can result in several common side effects including fever and injection-site pain. Patients are often advised to use acetaminophen or other over-the-counter pain medications as needed. The need for pharmacological interventions during Interferon beta treatments and the therapeutic role of IRF9 inhibition has been documented in a 2010 study by Farnsworth et al., which states: “The depression of genes that can positively or negatively regulate IFN-signalling in addition to downstream IFN targeted genes suggest that lower overall signal transduction occurs when IFN-β is administered with Acetaminophen”. Acetaminophen is an IRF9 inhibitor.

Neuropathic pain (NP) is a significant and disabling clinical problem with very few therapeutic treatment options available. Treatment options currently include medicines, like prescription pain relievers or anticonvulsants and antidepressants, as well as electrical stimulation and other techniques—none has resolved or cured this painful medical condition. Initial insults to the nervous system, such as spinal cord injury (SCI), are often compounded by secondary mechanisms such as inflammation (Schomberg et al., 2012).

Nonsteroidal antiinflammatory drugs (NSAIDs) are used to assist in the management of various chronic pain syndromes (Herndon et al., 2008). As a group, these medications are the most widely used medications in the world (Dugowson et al., 2006). Pain relief and decreased inflammation produced by NSAIDs result from suppression of the COX function of prostaglandin H synthase and the consequent formation of prostaglandin E2 (PGE(2)) and prostaglandin 12 (prostacyclin). Both cyclooxygenase-1 and -2 are expressed in the spinal cord, and the spinal COX product PGE(2) contributes to the generation of central sensitization upon peripheral inflammation. Further, spinal COX inhibition is also considered an important mechanism of antihyperalgesic pain treatment (Telleria-Diaz et al., 2010).

However, therapeutic utility of NSAIDs is limited by undesirable adverse effects including cardiovascular and gastrointestinal toxicity, for example producing ulcers (Dugowson et al., 2006; Herndon et al., 2008). On top of the widely known adverse effects a recent study by Farnsworth et al. 2010 have identified Acetaminophen as an inhibitor of the immune response system via Interferon Regulatory Factor 9 inhibition.

Similarly, here are practically no effective treatment options for neurodegenerative diseases, Including Alzheimer's diseases. There are currently five medications which are FDA-approved for the treatment of Alzheimer's disease. All five are cholinesterase inhibitors: tacrine (COGNEX®; Sciele), donepezil (ARICEPT®; Pfizer), rivastigmine (EXELON®; Novartis), galantamine (RAZADYNE®), and memantine (NAMENDA®; Forest Ortho-McNeil-Janssen). Donepezil, rivastigmine, and galantamine are successors to tacrine, a first-generation compound rarely prescribed because of the potential for hepatotoxicity. Donepezil (Aricept) and galantamine. All of the above-mentioned medications fail to produce detectable behavioral and cognitive improvements.

As far as available medications to treat chronic Inflammation the options to patients are few and inadequate. Metformin is commonly used in the treatment of type II diabetic patients with dyslipidemia and low-grade inflammation. The anti-inflammatory activity of Metformin is evident by reductions in circulating TNF-alpha, IL-1beta, CRP, and fibrinogen in these patients. Statins are anti-inflammatory as they reduce multiple circulating and cellular bio mediators of inflammation. This pleiotropic effect appears to contribute in part to the reduction in cardiovascular events. Corticosteroids also prevent several mechanisms involved in inflammation. Glucocorticoids are prescribed for inflammatory conditions including inflammatory arthritis, systemic lupus, sarcoidosis, and asthma.

Clearly, there is a lack in effective medications for treatment of cancer.

The present inventor has identified compounds and therapeutic compositions which enhance the expression and/or function of microRNA 22 and methods of use thereof for the treatment of various diseases.

### Embodiment of the Present Disclosure: Molecules, Compounds and Therapeutic Compositions, and Methods of Use Thereof

In some embodiments, the compounds of the present disclosure include:

The group of Interferon Signalling Regulators includes the following groups: microRNA 22 enhancers- IRF9 inhibitors, microRNA 22 enhancers- IRF9 agonists, microRNA 22 enhancers, microRNA 22 inhibitors, IRF9 agonists.

Group of microRNA 22 enhancers- IRF9 inhibitors comprising: alsterpaullone, azacitidine, azacytidine, camptothecin, helveticoside, lycorine, acetaminophen, Coxistac, Coumestrol, aspirin.

Group of microRNA 22 enhancers- IRF9 agonists comprising: 8-azaguanine, bepridil, azacyclonol, chlorprothixene, desipramine, fendiline, iopamidol, prenylamine, iopamidol, prochlorperazine, protriptyline, tetrandrine, suloctidil, tribenoside, cyclosporin.

Group of IRF9 agonists comprising: Retinoic acid, Medroxyprogesterone acetate, Copper sulfate, ursolic acid, ethyl methanesulfonate, 5-Fluorouracil, ellagic acid, Demecolcine, estradiol, 2-Methyl choline, benzo[a]pyrene, Tetradioxin, Tamibarotene, acetohexamide, amiloride, azaperone, benfluorex, benzbromarone, bupropion, cephaeline, cimetidine, clioquinol, clorgiline, diloxanide, drofenine, hexetidine, hydrocotarnine, lysergol, mephentermine, metoprolol, mifepristone, niflumic acid, Norethisterone, oxybutynin, pimozide, propofol, pyrithyldione, remoxipride, skimmianine terfenadine.

Group of microRNA 22 enhancers comprising: 7-aminocephalosporanic acid, ajmaline, alimemazine, alprostadil, alvespimycin, ambroxol, amitriptyline, amodiaquine, amoxapine, amprolium, anisomycin, apigenin, astemizole, benperidol, bergenin, betazole, botulin, bisacodyl, bufexamac, bupivacaine, butein, C-75, calcium pantothenate, calmidazolium , canavanine, capsaicin, carbinoxamine, carcinine, carmustine, cefuroxime, cetirizine, chlortetracycline, cycloheximide, cinchonidine, clenbuterol, clindamycin, clofazimine, clomifene, clopamide, cloperastine, clotrimazole, colchicine, colistin , conessine, corbadrine, corticosterone , corynanthine, CP-645525-01, cyclizine, cyclopenthiazide, cyproheptadine , danazol, daunorubicin, deptropine, diflunisal, digoxigenin, digoxin, dilazep, dimethadione, dipyridamole, disulfiram, doxylamine, dydrogesterone, ebselen, econazole, ellipticine, enilconazole, epitiostanol, etacrynic acid, etoposide, F0447-0125, fenbendazole, fluorometholone, fluphenazine, fluspirilene, flutamide, fosfosal, fulvestrant, galantamine, geldanamycin, gliclazide, halofantrine, hecogenin, homochlorcyclizine, hycanthone ,hydroxyachillin, idoxuridine, imipenem, indoprofen, irinotecan, isoconazole, isoetarine, isotretinoin, isoxsuprine, lanatoside C, lasalocid, letrozole, lomustine, loperamide, lumicolchicine, LY-294002, maprotiline, mebendazole, mebhydrolin, mefloquine, menadione, mepacrine, meptazinol, mestranol, metergoline, methylbenzethonium chloride, meticrane, metitepine, metixene, MG-262, miconazole, midecamycin, minaprine, mitoxantrone, molindone, mometasone, monorden, mycophenolic acid, naftopidil, naringin, netilmicin, niclosamide, nifuroxazide, nisoxetine, norcyclobenzaprine, nortriptyline, orciprenaline, ouabain, oxetacaine, oxetacaine, oxyphenbutazone, pancuronium bromide, parthenolide, penbutolol, pentoxyverine, pepstatin, perhexiline, perphenazine, phenazone, phenoxybenzamine, phensuximide, pimethixene, piperine, piperlongumine, pivmecillinam, pizotifen, podophyllotoxin, prednicarbate, Prestwick-983, proadifen, probenecid, procarbazine, procyclidine, profenamine, promazine, propantheline bromide, proscillaridin, pyrimethamine, pyrvinium, quinisocaine, quinpirole, raloxifene, reserpine, rilmenidine, rimexolone, roxithromycin, S-propranolol, salsolidin, sanguinarine, semustine, seneciphylline, simvastatin, spectinomycin, spiperone, SR-95531, STOCK1N-, 5215, strophanthidin, sulfanilamide, sulfaphenazole, sulfathiazole, tacrine, tamoxifen, tanespimycin, thapsigargin, theophylline, thioguanosine, thioridazine, thiostrepton, tonzonium bromide, tretinoin, triamterene, trichlormethiazide, trichostatin A. withaferin A, Coumarin, 1α,25-(OH)2D3, 1-hydroxycholecalciferol, choline, 3,3-diphenyl-N-(1-phenylethyl)propan-1-amine (SENSE-33) (R)-enantiomer of 3,3-diphenyl-N-(1-phenylethyl)propan-1-amine (SENSE-33R) (S)-enantiomer of 3,3-diphenyl-N-(1-phenylethyl)propan-1-amine (SENSE-33S) 2-[(1R)-1-phenyl-3-[(1-phenylethyl)amino]propyl]phenol 2-[(1S)-1-phenyl-3-[(1-phenylethyl)amino]propyl]phenol.

Group of microRNA 22 inhibitors comprising: Primaquine, Biotin, AR-A014418, valproic acid, Urapidil, quercetin, MS-275, nicotinic acid, Hydrocortisone, Naftidrofuryl, Apomorphine, Flucytosine, GW-8510 ,staurosporine, sirolimus, MS-275, Meglumine, (+)-chelidonine, H-7, doxorubicin.

In some embodiments, the present disclosure provides pharmaceutical compositions. Each said pharmaceutical composition comprises a therapeutically effective amount of at least one compound or molecule selected from the compounds of the present disclosure, wherein the term “compounds of the present disclosure” refers to all of the compounds listed, illustrated, described or mentioned in the present disclosure, and a compound selected from the compounds of the present disclosure may be any compound listed, illustrated, described or mentioned in the present disclosure.

In one aspect, the present disclosure relates to pharmacological methods and compositions for the treatment of chronic inflammatory diseases by altering the expression of the Interferon signalling pathway in a cell comprising contacting the cell with a molecule that binds to, and alters, expression of one or more of Interferon Regulatory Factors IRF1. IRF2, IRF3, IRF4, IRF5, IRF6, IRF7, IRF8, IRF9 or homologues thereof, or the activity of an encoded protein, in the cell.

In one embodiment of the present disclosure, the molecule is microRNA 22 encoded by a nucleic acid selected from the group consisting of siRNA, miRNA, antisense DNA, antisense RNA, triplex forming oligonucleotide, ribozyme, or external guide sequence for ribonuclease P and antisense DNA/RNA hybrids.

In another embodiment of the present disclosure the molecule is an IRF9 mRNA

In another embodiment of the present disclosure the molecule is an IRF9 mRNA

In another embodiment of the present disclosure the molecule is a microRNA targeting the IRF9 mRNA.

In another embodiment of the present disclosure the molecule is a vaccinia virus or a modified vaccinia virus.

In another embodiment of the present disclosure the molecule is selected from the group of Interferon Signalling Regulators.

### Synergistic Compositions

In some embodiments, the present disclosure provides a pharmaceutical composition comprising a therapeutically effective amount of a nucleic acid encoding miRNA22 and a therapeutically effective amount of IRF9 mRNA.

In some embodiments, the present disclosure provides a pharmaceutical composition comprising a therapeutically effective amount of a nucleic acid encoding MicroRNA22 and a therapeutically effective amount of IRF9 mRNA antagonist.

In some embodiments, the present disclosure provides a pharmaceutical composition comprising a therapeutically effective amount of a Vaccinia Virus and a therapeutically effective amount of a nucleic acid encoding miRNA22.

In some embodiments, the present disclosure provides a pharmaceutical composition comprising a therapeutically effective amount of a Vaccinia Virus and a therapeutically effective amount of a molecule enhancing the expression of IRF9 mRNA

In some embodiments, the present disclosure provides a pharmaceutical composition comprising a therapeutically effective amount of a Vaccinia Virus and a therapeutically effective amount of a compound selected from the group of the Interferon Signalling Regulators.

More specifically, in some preferred embodiments, the present disclosure provides a pharmaceutical composition comprising a vaccinia virus, or a modified vaccinia virus, and a compound enhancing the expression and or activity of IRF9 mRNA selected from the group of IRF9 agonists.

Any one of the pharmaceutical compositions according to the present disclosure may be used to treat a subject in need. More specifically, the present disclosure provides methods of treatment according to which any one of the pharmaceutical compositions according to the present disclosure may be administered to a subject in need. In some embodiments of the present disclosure, the subject in need has a disease or medical condition. Methods of treatment and diseases or medical conditions according to the present disclosure are further discussed below.

In other embodiments, the present disclosure relates to a pharmaceutical composition comprising a therapeutically effective amount of at least one microRNA 22 agonist and at least one microRNA 22 antagonist, wherein the microRNA 22 agonist and antagonist are selected from the compounds or molecules of the present disclosure. In some embodiments, said pharmaceutical composition is administered in an iterative manner to a subject in need for the treatment of a disease or a medical condition. In other embodiments, the present disclosure relates to a pharmaceutical composition comprising a therapeutically effective amount of at least one compound selected from the group of Interferon signalling regulators.

As an example of an embodiment of a pharmaceutical composition according to the present disclosure, said pharmaceutical composition comprised of therapeutically effective amounts of:
(i) a microRNA 22 agonist, and
(ii) a microRNA 22 antagonist.

In other embodiments, each compound category, i.e. agonist vs. antagonist, is in a separate pharmaceutical composition, so that one pharmaceutical composition comprises therapeutically effective amounts of at least one microRNA 22 agonist, while another pharmaceutical composition comprises therapeutically effective amounts of at least one microRNA 22 antagonist. Thus, for example, the two said compounds (i) and (ii) are comprised in separate pharmaceutical compositions and are administered, separately, in a sequential manner with a time interval in between administrations. Said time interval may be one year, one month or one week, as long as the said agonist and antagonist are not administered simultaneously, since the action of microRNA 22 agonist cancels the action of microRNA 22 antagonist.

In yet other embodiments, the present disclosure relates also to Fendiline and Aricept. The present inventor has surprisingly discovered that Fendiline upregulates the long non-coding RNA (lncRNA) MIR22HG. Thus, as Fendiline upregulates the expression of MIR22HG, the expression of MICRORNA 22 is up-regulated as well.

Accordingly, in some embodiments the present disclosure provides a pharmaceutical composition comprising therapeutically effective amounts of Aricept and a microRNA 22 agonist selected from the microRNA 22 agonists of the instant disclosure for the treatment of diseases and medical conditions.

The present disclosure is also directed to methods for synergistically enhancing the activity of pharmaceutical agents. The efficacy of active components can sometimes be improved by addition of other (active) ingredients. More rarely, the observed efficacy of a combination of ingredients can be significantly higher than what would be expected from the amounts of the individual ingredients used, thus indicating potentiation of the activity of the components of the combination. Generally, the active component/s are any one of the compounds or molecules mentioned, listed illustrated, or described in the present disclosure. In some embodiments, the present disclosure provides a pharmaceutical composition comprising therapeutically effective amounts of at least one of the compounds or molecules of the present disclosure and at least one antibiotic. In some embodiments, the active components of the said pharmaceutical composition comprising one of the compounds or molecules of the present disclosure and at least one antibiotic act synergistically as a medicine for the treatment of diseases or medical conditions.

Non limiting examples for antibiotics which may be used in the presently disclosed pharmaceutical compositions include fungal antibiotics such as penicillins, cephalosporins griseofulvin ciclosporin, actinomycetal antibiotics such as streptomycin and related aminoglycoside antibiotics chloramphenicol, the tetracycline antibiotics, macrolide antibiotics, rifamycin antibiotics polyene antibiotics, azomycin vancomycin, teicoplanin thienamycin clavulanic acid, cytotoxic antibiotics from streptomycetes such as bleomycin, anthracycline antibiotics and monobactam antibiotics such as sulfazecin.

Other, particular, embodiments of presently disclosed methods of treatments are discussed below.

In one aspect the present disclosure relates to microRNA 22 encoded by a nucleic acid selected from the group consisting of siRNA, miRNA, antisense DNA, antisense RNA, triplex forming oligonucleotide, ribozyme, or external guide sequence for ribonuclease P, and antisense DNA/RNA hybrids for the treatment of diseases related to chronic inflammation, autoimmune disorders, and Pain.

In one embodiment, the present disclosure relates to the use of vaccinia virus, or modified vaccinia virus, for the treatment of diseases related to chronic inflammation, autoimmune disorders, and Pain.

The inventor of the present disclosure has found that the combined administration of Vaccinia Virus in conjunction with certain compounds from the group of Interferon Signalling Regulators surprisingly and synergistically potentiates the anti inflammatory response of cells when the vaccinia virus is used for the treatment of diseases related to chronic inflammation, neurodegeneration, or pain.

The inventor of the present disclosure has also found that the administration of certain compounds of the group of Interferon Signalling Regulators in conjunction with NSAIDs surprisingly and synergistically potentiates the analgesic, anti inflammatory, and antiviral response of the cells.

In particular, the inventor of the present disclosure has found and demonstrated that the administration of compounds of the group of microRNA 22 enhancers-IRF9 agonists in conjunction with NSAIDs synergistically potentiates the anti inflammatory effect of the latter, indicating that the combination of a compounds of the group of microRNA 22 enhancers-IRF9 agonists and a NSAID reduces the doses of the latter needed to obtain effective reduction in inflammation.

In particular, the inventor of the present disclosure has found and demonstrated that the administration of compounds of the group of microRNA 22 enhancers-IRF9 agonists in conjunction with NSAIDs synergistically potentiates the anti inflammatory effect of the latter, indicating that the combination of a compounds of the group of microRNA 22 enhancers-IRF9 agonists and a NSAID reduces the doses of the latter needed to obtain effective reduction in analgesis when said NSAID's are used in Recombinant interferon treatment.

The inventor of the present disclosure has also found that the administration of certain compounds of Group of Interferon Signalling Regulators in conjunction with certain Antibiotics surprisingly and synergistically potentiates the cytotoxic activity of said antibiotics.

Any one of the pharmaceutical compositions of the present disclosure may be used in a method of treatment whereby said composition is administered to a subject in need to treat said subject. In some embodiments, said subject in need has a disease or a medical condition. By “Disease/s” or “medical condition/s” is meant any disease condition or disorder related to inflammatory diseases or disorders, cancer, neurodegenerative diseases, ageing, metabolism and the disturbances of the circadian rhythms. Non-limiting examples for diseases or medical conditions related to aging include atherosclerosis and cardiovascular disease, arthritis, cataracts, osteoporosis, type 2 diabetes, hypertension and Alzheimer's disease, decision making impairment, cognitive decline, age-related cognitive decline, memory loss, mild memory loss, age related memory loss, ageing, apathy, depression, mild behavioral impairment, Mild Cognitive Impairment, cognitive decline, pain, neurodegenerative disorders, orthostatic hypotension, cholinergic system dysfunction, cholesterol homeostasis, neuropathic pain and neuropsychiatric symptoms.

The terms “inflammatory disease/s”, “inflammatory disorder/s” or “disease/s related to inflammation”, and similar meaning term/s, are used interchangeably herein. By “inflammatory disease/s”, “inflammatory disorder/s” or “disease/s related to inflammation”, and similar meaning term/s, is meant, without limitation, allergic reactions, myopathies, abnormal inflammation, cancer, atherosclerosis, ischemic heart disease, Acne vulgaris, Asthma, Autoimmune “inflammatory disease/s”, “inflammatory disorder/s” or “disease/s related to inflammation”diseases, Autoinflammatory diseases, Celiac disease, Chronic prostatitis, Colitis, Diverticulitis, Glomerulonephritis, Hidradenitis suppurativa, Hypersensitivities, Inflammatory bowel diseases, Interstitial cystitis, Lichen planus, Mast Cell Activation Syndrome, Mastocytosis, Otitis, Pelvic inflammatory disease, Reperfusion injury, Rheumatic fever, Rheumatoid arthritis, Rhinitis, Sarcoidosis, Transplant rejection, and Vasculitis.

By “neurological disorder” is meant any disease, condition and disorder of the central nervous system. Neurological disorders according to the present disclosure are, but not limited to, Alzheimer's disease, Parkinson's Disease, amyotrophic lateral sclerosis, epilepsy, autism, rett syndrome, Huntington's Disease, stroke, spinal cord injury, traumatic brain injury, Lewy body dementia, multiple sclerosis, Pick's disease, Niewmann-Pick disease, Creutzfeld-Jakob disease, Guillain-Barre syndrome, Bell's palsy, diabetic neuropathy, amyotrophic lateral sclerosis, amyloid angiopathy, cerebral amyloid angiopathy, systemic amyloidosis, Petit Mal Epilepsy, Temporal Lobe Epilepsy, Post-Traumatic Epilepsy, Generalized Seizure Disorder, all forms of Epilepsy, Postherpetic Neuralgia, Viral Meningitis, Viral Encephalitis, Acute Poliomyelitis, Postpolio Syndrome, HIV Dementia , HIV-associated Myelopathy , HIV-associated Peripheral Neuropathy, HTLV-1 Myelopathy, Influenza, Trigeminal Neuralgia, Reyets Syndrome, Meniere's Disease, Reflex Sympathetic Dystrophy , Cerebrovascular Disease, Prophylaxis of Brain Injury caused by Stroke, Chronic Inflammatory Demyelinating Polyneuropathy, major depression, anxiety-related disorders, post-traumatic stress disorder, supranuclear palsy, feeding disorders, fibromyalgia , single episode depression, recurrent depression, child abuse induced depression, and postpartum depression; dysthemia; bipolar disorders; cyclothymia; fatigue syndrome; stress-induced headache, hereditary cerebral hemorrhage with amyloidosis of the Dutch type, inclusion body myositis, mild cognitive impairment, complications due to stroke, head trauma, or spinal injury, or other injuries to the brain, peripheral nervous, central nervous or neuromuscular system, acute spinal cord and brain injury, demyelinating diseases such as multiple sclerosis, pain, acute pain, chronic pain, neuropathic pain, acute neuropathic pain, chronic neuropathic pain, migraine, post herpetic neuralgia, neuritis, temporomandibular disorder, myofascial pain, back pain, diabetic neuropathy, numbness, tingling sensation, trauma or injury to a nerve, allodynia, dystonia, Leber's hereditary optic neuropathy (LHON), schizophrenia.

By “Neuropathic pain” is meant pain associated with damage or permanent alteration of the peripheral or central nervous system. Clinical manifestations of neuropathic pain include a sensation of burning or electric shock, feelings of bodily distortion, allodynia and hyperpathia.

Non-limiting examples for diseases or medical conditions related to metabolism diabetes, type 2 diabetes mellitus, type 1 diabetes mellitus, ulcers, irritable bowel syndrome, Crohn's disease, spastic colon, diarrhea, and post-operative ilius, nausea, emesis, ulcers, and constipation, coronary heart disease, hypertension, angina pectoris, myocardial infarction, stroke, skin and connective tissue disorders, foot ulcerations, metabolic acidosis, arthritis, osteoporosis and in particular conditions of impaired glucose tolerance, maturity-onset diabetes of the young (MODY), and gestational diabetes), satiety, endocrine deficiencies of aging, and any of their associated complications (e.g., Syndrome X, diabetic retinopathy, diabetic nephropathy, diabetic neuropathy, peripheral vascular disease, hyperlipidemia, hypertension, atherosclerosis, and coronary heart disease), hyperlipidemia, fatty liver disease, cachexia, obesity, atherosclerosis and arteriosclerosis, mitochondrial cytopathies such as “mitochondrial encephalopathy, lactic acidosis, and stroke” (MELAS), and “myoclonic epilepsy ragged red fiber syndrome” (MERRF), NARP (Neuropathy; Ataxia; Retinitis Pigmentosa), MNGIE (Myopathy and external opthalmoplegia, neuropathy; gastro-intestinal encephalopathy, Kearns-Sayre disease, Pearson's Syndrome, PEO (Progressive External Opthalmoplegia), congenital muscular dystrophy with mitochondrial structural abnormalities, Wolfram syndrome, Diabetes Insipidus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, and maternally inherited diabetes with deafness (MIDD), mitochondrial DNA depletion syndrome.

Non-limiting examples for diseases or medical conditions related to circadian rhythms include continuous or occasional disruption of sleep patterns, Delayed Sleep Phase Disorder, Advanced Sleep Phase Disorder, Jet Lag, Shift Work Disorder and Narcolepsy.

Preferably, the cancerous disease to be treated or prevented in the above aspects of the present disclosure is selected from the group consisting of: acute monocytic leukemia, acute myelogenous leukemia, acute myelomonocytic leukemia, acute promyelocytic leukemia, adult T-cell leukemia, adult T-cell lymphoma, astrocytoma, atypical carcinoid lung cancer, basal cell carcinoma, B-acute lymphocytic leukemia, B-cell acute lymphoblastic leukemia/lymphoma, Bladder cancer, brain cancer, breast cancer, bronchial cancer, Burkitt's lymphoma, cancer of the bile duct, cancer of unknown primary origin, cervix cancer, chronic myeloproliferative disorder, colon cancer, diffuse large cell lymphoma, endometrial cancer, ependymoma, esophageal cancer, gastric cancer, glioma, glioblastoma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, melanoma, Multiple myeloma, myelodysplastic syndrome, nasopharygeal cancer, neuroblastoma, NK cell tumor, nonHodgkin's lymphoma, oesophageal squamous cell carcinoma, osteosarcoma, ovarian cancer, pancreatic cancer, peripheral T-cell leukemia, primary plasma cell leukemia, prostate cancer, renal clear cell carcinoma, retinoblastoma, rhabdomyosarcoma, sarcoma, small cell lung carcinoma, T-cell acute lymphoblastic leukemia, T-cell acute lymphoblastic lymphoma, testicular cancer, thymoma, urachal cancer, uterine cancer, vaginal cancer, and combinations thereof.

The present disclosure is also directed to methods for treating conditions or diseases related to pain related to microRNA 22 activity or expression, particularly pain, acute pain, chronic pain, neuropathic pain, acute neuropathic pain, chronic neuropathic pain, migraine, post herpetic neuralgia, neuritis, temporomandibular disorder, myofascial pain, back pain, diabetic neuropathy, numbness, tingling sensation, trauma or injury to a nerve, or in variety of rheumatologic conditions, such as ankylosing spondylitis, rheumatoid arthritis and Osteoarthritis which involves at least intermittent inflammation.

Any of the pharmaceutical compositions of the present disclosure may also comprise a pharmaceutically accepted salt and/or at least one excipient. In some embodiments, the compositions further comprise one or more pharmaceutically acceptable salts or excipients. Pharmaceutically accepted salts or excipients are further discussed below.

Any of the compounds or molecules employed in the methods and compositions of the present disclosure may contain one or more asymmetrically-substituted carbon or nitrogen atoms, and may be isolated in optically active or racemic form. Thus, all chiral, diastereomeric, racemic form, epimeric form, and ail geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The compounds can be formulated as a mixture of one or more diastereomers. Alternatively, the diastereomers can be separated and one or more of the diastereomers can be formulated individually. The chiral centers of the compounds of the present disclosure can have the S or the R configuration, as defined by the IUPAC 1974 Recommendations. For example, mixtures of stereoisomers may be separated using the techniques taught in the Examples section below, as well as modifications thereof.

Atoms making up the compounds or molecules of the present disclosure are intended to include all isotopic forms of such atoms. Compounds or molecules employed in the present disclosure include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include 13C and 14C. Similarly, it is contemplated that one or more carbon atom(s) of a compound or molecules of the present disclosure may be replaced by a silicon atom(s). Furthermore, it is contemplated that one or more oxygen atom(s) of a compound or molecules of the present disclosure may be replaced by a sulfur or selenium atom(s).

Compounds or molecules employed in the present disclosure may also exist in prodrug form. Since prodrugs are known to enhance numerous desirable qualities of pharmaceuticals (e.g., solubility, bioavailability, manufacturing, etc.), the compounds or molecules employed in some methods of the disclosure may. if desired, be delivered in prodrug form. Thus, the disclosure contemplates prodrugs of compounds or molecules of the present disclosure as well as methods of delivering prodrugs. Prodrugs of the compounds or molecules employed in the disclosure may be prepared by modifying functional groups present in the compound or molecules in such a way that the modifications are cleaved, either in routine manipulation or in vivo, to the parent compound or molecule. Accordingly, prodrugs include, for example, compounds or molecule described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

A subject in need according to the present disclosure is a subject having, without limitation, any of the following disorders, diseases or conditions: inflammatory diseases or disorders, cancer, neurodegenerative diseases, ageing, metabolism, disturbances of the circadian rhythms, aging, aging related diseases or conditions, metabolism-related diseases or conditions, disturbances of the circadian rhythms, disturbances of the circadian rhythms-related diseases and conditions, a neurological disorder, dementia, a nervous system-related tumor, cancer, diabetes, brain cancers, glioblastomas, retinoblastomas, pediatric neuroblastoma, glioblastoma, Brain Trauma Injuries, Metabolic diseases, obesity, Renal Fibrosis, Myelination disorder, Multiple Sclerosis, chronic wounds, non-healing wounds, skin disorders, or is in need of increased stem cell regeneration

The compositions of the disclosure may include one or more of these compounds or molecules, which may be present in amount(s) of at least 1, 5, 10, 25, 50, 100, 250, 500, or 750 μg; at least 1, 2, 5, 10, 25, 50, 100, 250, 500, or 750 mg; or at least 1, 2, 5, 10, 15, 20, 25, 50, or 100 g, or any range between these values. The compositions may include at least 0.01%, 0.05%, 0.1%, 0.5%, 1%, 2%, 3%, 5%, 8%, 10%, 15%, 20%, 25%, 40%, 50%, 60%, 75%, 85%, 90%, 95%, 98%, or 99% of the lipid(s), or any range between these values. In certain embodiments, the compositions include less than 99%, 98%, 95%, 90%, 85%, 75%, 60%, 50%, 40%, 25%, 20%, 15%, 8%, 5%, 3%, 2%, 1%, or 0.5% of the lipid(s). In compositions containing two or more compounds or molecules, the ratio between any two of the compounds or molecules present may be at least 1000:1, 500:1, 100:1, 50:1, 25:1, 10:1, 5:1, 3:1, 2:1, 1:1, 1:2, 1:3, 1:5, 1:10, 1:25, 1:50, 1:100, 1:500, or 1:1000, or any range between these values.

The compound/s, molecule/s, or composition/s of the disclosure are given on a per diem basis but should not be interpreted as necessarily being administered on a once daily frequency. Indeed, the compositions, compound, molecules, salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. In some situations, a single dose may be administered, but more typically administration is according to a regimen involving repeated dosage over a treatment period. In such a regimen the daily dose and/or frequency of administration can, if desired, be varied over the course of the treatment period, for example introducing the subject to the compound, molecules, composition, salt or prodrug thereof at a relatively low dose and then increasing the dose in one or more steps until a full dose is reached. The treatment period is generally as long as is needed to achieve a desired outcome.

Any of the pharmaceutical compositions of the present disclosure, comprising pharmaceutically effective amounts of one or more of the compounds or molecules of the present disclosure (Active Pharmaceutical Ingredient/s) may also comprise a pharmaceutically acceptable vehicle, carrier or excipient. In fact, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present disclosure include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

The compositions of the disclosure may be in any of the forms described herein. In some embodiments, the composition is food that has been enriched with one or more of these compounds or molecules.

By “treating” is meant ameliorating at least one symptom of a condition or disease in a subject having the condition or disease (e.g., a subject diagnosed with a neurological disorder), as compared with an equivalent untreated control. Such reduction in the symptom (e.g., a reduction in blood glucose levels) is at least 5%, 10%, 20%, 40%, 50%, 60%, 80%, 90%, 95%, or 100%, as measured by any standard technique.

By “treating prophylactically” is meant to reduce the frequency of disease occurrence or severity of disease upon its onset by administering to the subject a therapeutic prior to onset of the disease. Prophylactic treatment can include disease prevention. Subjects at higher risk of developing metabolic disorders or IBD (e.g., risk factors described herein) may be treated prophylactically in the methods of the disclosure.

By “effective amount” or “therapeutically effective amount” is meant an amount of a compound or molecule required to treat, treat prophylactically, or reduce disease or disorder in a clinically relevant manner. For example, a effective amount of an active compound or molecule used to according to the present disclosure varies depending upon the manner of administration, the age, body weight, and general health of the subject. Ultimately, the prescribers will decide the appropriate amount and dosage regimen.

By “subject” is meant a human or non-human animal (e.g., a mammal).

By “increase” is meant an amount greater by at least 5%, 10%, 25%, 50%, 100%, 150%, 200%, 500%, or 1000%.

By “decrease” is meant an amount smaller by at least 5%, 10%, 25%, 50%, 75% or 100%.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

### REFERENCES

Chang, T.-C., Yu, D., Lee, Y.-S., Wentzel, E. A., Arking, D. E., West, K. M., Dang, C. V., Thomas-Tikhonenko, A., Mendell, J. T. Widespread microRNA repression by Myc contributes to tumorigenesis. Nature Genet. 40: 43-50, 2008.

E Dugowson, Carin & Gnanashanmugam, Priya. (2006). Nonsteroidal Anti-Inflammatory Drugs. Physical medicine and rehabilitation clinics of North America. 17. 347-54

Lánczky, András; Nagy, Ádám; Bottai, Giulia; Munkácsy, Gyöngyi; Szabó, András; Santarpia, Libero; Gy [symbol] rffy, Balázs (2016 Dec. 1). “miRpower: a web-tool to validate survival-associated miRNAs utilizing expression data from 2178 breast cancer patients”. Breast Cancer Research and Treatment. 160 (3): 439-446.

Farnsworth A, Flaman A S, Prasad S S, et al. Acetaminophen modulates the transcriptional response to recombinant interferon-beta. PLoS One. 2010; 5(6):e11031.

Ford E, Thanos D. The transcriptional code of human IFN-beta gene expression. Biochim Biophys Acta. 2010 March-April; 1799(3-4):328-36.

Michael Hecker, Madhan Thamilarasan 2, Dirk Koczan, Ina Schröder, Kristin Flechtner, Sherry Freiesleben, Georg Füllen, Hans-Jürgen Thiesen, Uwe Klaus Zettl. MicroRNA Expression Changes during Interferon-Beta Treatment in the Peripheral Blood of Multiple Sclerosis Patients. Int. J. Mol. Sci. 2013, 14(8), 16087-16110.

Herndon C M1, Hutchison R W, Berdine H J, Stacy Z A, Chen J T, Farnsworth D D, Dang D, Fermo J D. Management of chronic nonmalignant pain with nonsteroidal antiinflammatory drugs. Joint opinion statement of the Ambulatory Care, Cardiology, and Pain and Palliative Care Practice and Research Networks of the American College of Clinical Pharmacy. Pharmacotherapy. 2008 June; 28(6):788-805.

Jovicic A, Zaldivar Jolissaint J F, Moser R, Silva Santos Mde F, Luthi-Carter R. MicroRNA-22 (miR-22) overexpression is neuroprotective via general anti-apoptotic effects and may also target specific Huntington's disease-related mechanisms. PLoS One. 2013; 8(1):e54222.

Maniatis T1, Falvo J V, Kim T H, Kim T K, Lin C H, Parekh B S, Wathelet M G. Structure and function of the interferon-beta enhanceosome. Cold Spring Harb Symp Quant Biol. 1998; 63:609-20.

Geoffrey L. Smith, Callum Talbot-Cooper, Yongxu Lu. Chapter Fourteen—How Does Vaccinia Virus Interfere With Interferon? Editor(s): Margaret Kielian, Thomas C. Mettenleiter, Marilyn J. Roossinck, Advances in Virus Research, Academic Press, Volume 100, 2018, Pages 355-378.

Alvin Paul, Thean Hock Tang and Siew Kit Ng. Interferon Regulatory Factor 9 Structure and Regulation. Front. Immunol., 10 Aug. 2018.

Damon Polioudakis, Akshay A. Bhinge, Patrick J. Killion, Bum-Kyu Lee, Nathan S. Abell, Vishwanath R. Iyer. A Myc-microRNA network promotes exit from quiescence by suppressing the interferon response and cell-cycle arrest genes. Nucleic Acids Res. 2013 February; 41(4): 2239-2254.

Rauch I, Müller M, Decker T. The regulation of inflammation by interferons and their STATs. JAKSTAT. 2013; 2(1):e23820.

Dominic Schomberg, Mostafa Ahmed, Gurwattan Miranpuri, Julie Olson, and Daniel K Resnick, MD, MS. Neuropathic pain: role of inflammation, immune response, and ion channel activity in central injury mechanisms. Ann Neurosci. 2012 July; 19(3): 125-132.

Shengfeng Wan, Usama Ashraf, Jing Ye, Xiaodong Duan, Ali Zohaib, Wentao Wang, Zheng Chen, Bibo Zhu, Yunchuan Li, Huanchun Chen, Shengbo Cao. MicroRNA-22 negatively regulates poly(I:C)-triggered type I interferon and inflammatory cytokine production via targeting mitochondrial antiviral signaling protein (MAVS). Oncotarget. 2016 Nov. 22; 7(47): 76667-76683.

Shin H Y. Targeting Super-Enhancers for Disease Treatment and Diagnosis. Mol Cells. 2018 June; 41(6):506-514. doi: 10.14348/molcells.2018.

Telleria-Diaz A1, Schmidt M, Kreusch S, Neubert A K, Schache F, Vazquez E, Vanegas H, Schaible H G, Ebersberger A. Spinal antinociceptive effects of cyclooxygenase inhibition during inflammation: Involvement of prostaglandins and endocannabinoids. Pain. 2010 January; 148(1):26-35.

Thanos D, Maniatis T. Virus induction of human IFN beta gene expression requires the assembly of an enhanceosome. Cell. 1995 Dec. 29; 83(7):1091-100.

Jingyu Wang, Yuan Li, Meiman Ding, Honghe Zhang, Xiaoming Xu, and Jinlong Tang. Molecular mechanisms and clinical applications of miR-22 in regulating malignant progression in human cancer (Review). Int J Oncol. 2017 February; 50(2): 345-355.

Jianhua Xiong. Emerging Roles of MicroRNA-22 in Human Disease and Normal Physiology. Current Molecular Medicine. February 2012.

Zhang J, Yang Y, Yang T, Liu Y, Li A, Fu S, Wu M, Pan Z, Zhou W (2010). “microRNA-22, downregulated in hepatocellular carcinoma and correlated with prognosis, suppresses cell proliferation and tumourigenicity”. Br J Cancer. 103 (8): 1215-20.

1. A method for altering the Interferon signaling pathway in a cell comprising
contacting the cell with a compound or molecule that alters expression of one or more of Interferon Regulatory Factors IRF1. IRF2, IRF3, IRF4, IRF5, IRF6, IRF7, IRF8, IRF9 or homologues thereof, or the activity, of an encoded protein, in the cell.

2. The method of claim 1 wherein the compound or molecule is inhibiting the interferon signaling and is selected from one of the groups, lists, or descriptions of the present disclosure.

3. The method of claim 1 wherein the inhibitory molecule is: [symbol]
A modified vaccinia virus synthetically modified by the presence, in a non-essential region of the vaccinia genome, of DNA not naturally occurring in vaccinia virus, wherein said DNA is expressed in a host by the production of a protein, and wherein said protein is an antigen.

5. A method according to claim 2 wherein the molecule is an enhancer of the Interferon signaling pathway in a cell and enhances the expression of Interferon Regulatory Factor 9 (IRF9) or homologues thereof, or the activity of an encoded protein, in the cell.

6. The method of claim 1 for increasing survivability of a cell comprising
administering to the cell an amount of a compound or molecule that inhibits the expression of Hn-I 4, lin-28, lin-42, egl-35 or homologues thereof, or the activity of an encoded protein, in the cell.

7. The method of claim 6, wherein the compound or molecule is selected from one of the groups lists, or descriptions of the present disclosure.

8. The method of claim 1 wherein the compound or molecule is administered to, or expression is increased in, the cells of, an individual for treatment or prevention of an age-related disorder or premature aging, and wherein the compound or molecule is selected from one of the groups lists, or descriptions of the present disclosure.

9. The method of claim 8 wherein the age-related disorder is selected from the group consisting of Alzheimer's, Parkinson's, diabetes, dementia, atherosclerosis, arthritis, stroke, high blood pressure, and heart disease.

10. The method of claim 9 wherein the individual is a human.

11. The method according to claim 1 wherein the compound or molecule is an enhancer of the Interferon signaling pathway in a cell and enhances the expression of Interferon Regulatory Factor 9 (IRF9) or homologues thereof, or the activity of an encoded protein, in the cell, wherein the compound or molecule is selected from one of the groups lists, or descriptions of the present disclosure.

12. The method of claim 11 wherein the inhibitory molecule is administered to, or expression is increased in, the cells of, an individual for treatment or prevention of an inflammatory disorder.

13. The method of claim 12 wherein the inflammatory disorder is selected from the group consisting of allergic reactions, myopathies, abnormal inflammation, atherosclerosis, ischemic heart disease, Rheumatic fever, and Rheumatoid arthritis.

14. The method of claim 13 wherein the individual is a human.

15. The method of claim 1 for increasing survivability of a cell comprising administering to the cell an amount of a compound that inhibits the expression of Interferon Regulatory Factors or homologues thereof, or the activity of an encoded molecule, in the cell, wherein the compound or molecule is selected from one of the groups lists, or descriptions of the present disclosure.

16. The method of claim 15 wherein the inhibitory molecule is administered to, or expression is increased in, the cells of, an individual for treatment or prevention of cancer.

17. The method of claim 17 wherein the individual is a human.
